SEQUENCE LISTING (4 sequences)

SEQ ID NO: 1 - DNA, 1173 bp, Homo sapiens
atgaattcactcagtgaagccaacaccaagttcatgttcgacctgttccaacagttcagaaaatcaaaagagaacaacatcttctattcccctatcagcatcacatcagcattagggatggtcctcttaggagccaaagacaacactgcacaacagattaagaaggttcttcactttgatcaagtcacagagaacaccacaggaaaagctgcaacatatcatgttgataggtcaggaaatgttcatcaccagtttcaaaagcttctgactgaattcaacaaatccactgatgcatatgagctgaagatcgccaacaagctcttcggagaaaaaacgtatctatttttacaggaatatttagatgccatcaagaaattttaccagaccagtgtggaatctgttgattttgcaaatgctccagaagaaagtcgaaagaagattaactcctgggtggaaagtcaaacgaatgaaaaaattaaaaacctaattcctgaaggtaatattggcagcaataccacattggttcttgtgaacgcaatctatttcaaagggcagtgggagaagaaatttaataaagaagatactaaagaggaaaaattttggccaaacaagaatacatacaagtccatacagatgatgaggcaatacacatcttttcattttgcctcgctggaggatgtacaggccaaggtcctggaaataccatacaaaggcaaagatctaagcatgattgtgttgctgccaaatgaaatcgatggtctccagaagcttgaagagaaactcactgctgagaaattgatggaatggacaagtttgcagaatatgagagagacacgtgtcgatttacacttacctcggttcaaagtggaagagagctatgacctcaaggacacgttgagaaccatgggaatggtggatatcttcaatggggatgcagacctctcaggcatgaccgggagccgcggtctcgtgctatctggagtcctacacaaggcctttgtggaggttacagaggagggagcagaagctgcagctgccaccgctgtagtaggattcggatcatcacctacttcaactaatgaagagttccattgtaatcaccctttcctattcttcataaggcaaaataagaccaacagcatcctcttctatggcagattctcatccccgtag

SEQ ID NO: 2 - DNA, 1173 bp, Homo sapiens
atgaattcactcagtgaagccaacaccaagttcatgttcgatctgttccaacagttcagaaaatcaaaagagaacaacatcttctattcccctatcagcatcacatcagcattagggatggtcctcttaggagccaaagacaacactgcacaacaaattagcaaggttcttcactttgatcaagtcacagagaacaccacagaaaaagctgcaacatatcatgttgataggtcaggaaatgttcatcaccagtttcaaaagcttctgactgaattcaacaaatccactgatgcatatgagctgaagatcgccaacaagctcttcggagaaaagacgtatcaatttttacaggaatatttagatgccatcaagaaattttaccagaccagtgtggaatctactgattttgcaaatgctccagaagaaagtcgaaagaagattaactcctgggtggaaagtcaaacgaatgaaaaaattaaaaacctatttcctgatgggactattggcaatgatacgacactggttcttgtgaacgcaatctatttcaaagggcagtgggagaataaatttaaaaaagaaaacactaaagaggaaaaattttggccaaacaagaatacatacaaatctgtacagatgatgaggcaatacaattcctttaattttgccttgctggaggatgtacaggccaaggtcctggaaataccatacaaaggcaaagatctaagcatgattgtgctgctgccaaatgaaatcgatggtctgcagaagcttgaagagaaactcactgctgagaaattgatggaatggacaagtttgcagaatatgagagagacatgtgtcgatttacacttacctcggttcaaaatggaagagagctatgacctcaaggacacgttgagaaccatgggaatggtgaatatcttcaatggggatgcagacctctcaggcatgacctggagccacggtctctcagtatctaaagtcctacacaaggcctttgtggaggtcactgaggagggagtggaagctgcagctgccaccgctgtagtagtagtcgaattatcatctccttcaactaatgaagagttctgttgtaatcaccctttcctattcttcataaggcaaaataagaccaacagcatcctcttctatggcagattctcatccccatag

SEQ ID NO: 3 - protein, 390 aa, Homo sapiens [sequence omitted]

SEQ ID NO: 4 - protein, 390 aa, Homo sapiens
MNSLSEANTKFMFDLFQQFRKSKENNIFYSPISITSALGMVLLGAKDNTAQQISKVLHFDQVTENTTEKAATYHVDRSGNVHHQFQKLLTEFNKSTDAYELKIANKLFGEKTYQFLQEYLDAIKKFYQTSVESTDFANAPEESRKKINSWVESQTNEKIKNLFPDGTIGNDTTLVLVNAIYFKGQWENKFKKENTKEEKFWPNKNTYKSVQMMRQYNSFNFALLEDVQAKVLEIPYKGKDLSMIVLLPNEIDGLQKLEEKLTAEKLMEWTSLQNMRETCVDLHLPRFKMEESYDLKDTLRTMGMVNIFNGDADLSGMTWSHGLSVSKVLHKAFVEVTEEGVEAAAATAVVVVELSSPSTNEEFCCNHPFLFFIRQNKTNSILFYGRFSSP

(19) United States
(12) Patent Application Publication
(10) Pub. No.: US 2008/0057520 A1
(43) Pub. Date: Mar. 6, 2008

(54) IMMUNOASSAYS FOR SPECIFIC DETERMINATION OF SCCA ISOFORMS

(75) Inventors: Eva Rojer, Goteborg (SE); Olle Nilsson, Goteborg (SE)

Correspondence Address: GAUTHIER & CONNORS, LLP, 225 FRANKLIN STREET, SUITE 2300, BOSTON, MA, 02110, US

(73) Assignee: CANAG DIAGNOSTICS AB, Goteborg (SE)

(21) Appl. No.: 11/765,725

(22) Filed: Jun. 20, 2007

Related U.S. Application Data
(62) Division of application No. 10/652,705, filed on Aug. 29, 2003.
(60) Provisional application No. 60/409,484, filed on Sep. 10, 2002.

(30) Foreign Application Priority Data
Sep. 10, 2002 (SE) 0202702-7

(51) Int. Cl. C07K 16/00; G01N 33/574

(52) U.S. Cl. 435/7.23; 530/300; 530/387.3; 530/388.1

(57) ABSTRACT

The present invention relates to monoclonal antibodies capable of distinguishing squamous cell cancer antigens, SCCA, in either free or complex bound forms, preferably antigens SCCA1 and SCCA2, as well as hybridomas recognizing such antibodies, method for diagnosing SCC, as well as diagnostic kits for detecting SCCAs.

### PRIORITY INFORMATION

This application is divisional of U.S. patent application Ser. No. 10/652,705, filed on Aug. 29, 2003 which claims priority to U.S. Provisional Patent Application No. 60/409,484, filed on Sep. 10, 2002 and to Swedish Patent Application No. 0202702, filed on Sep. 10, 2002, all of which are incorporated herein by reference in their entirety.

### BACKGROUND OF THE INVENTION

1. Field of the Invention

The present invention relates to the specific determination of different isoforms of SCCA and the use of the serological concentration of the different isoforms and ratio between them as a means of diagnosis of cancer.

2. Brief Description of the Art

Squamous cell carcinoma antigen (SCCA) is a serological marker for squamous cell carcinomas (SCC) of the uterine, cervix, lung, head and neck, vulva, and esophagus (1, 2). It was originally purified in the end 70-ties by Kato and coworkers from the TA-4 complex from human cervical squamous cell carcinoma, with a molecular weight of 42-48 kDa (1, 3). Electrophoresis of the TA-4 complex revealed more than 10 fractions and iso-electric focusing of the antigen suggested two subfractions, an acidic (pI<6.25) and a neutral (pI≧6.25) isoform (4).

Cloning of the cDNA of SCCA shows that it belongs to the family of serine protease inhibitors (serpins) (6). Further cloning of the genomic region on chromosome 18q21.3 revealed two tandemly arrayed genes (7). The more telomeric one, the original SCCA, was designated SCCA1, whereas the more centromeric one was designated SCCA2 (FIG. 1). They both contain eight exons and the putative intron-exon boundaries, splice sites, initiation codons, and terminal codons are identical. They are 98% identical at the nucleotide level and 92% identical at the amino acid level. The deduced pI values of the SCCA1 and SCCA2 gene products show that the neutral isoform are coded by SCCA1 and the acidic isoform by SCCA2.

In humans the serpins map to one of two chromosomal clusters. PI6, PI9 and ELNAH2 map to 6p25, whereas PI8, Bomapin, PAI2, SCCA1, SCCA2, Headpin and Maspin map to 18q21.3 (FIG. 1)(7-12). These clusters are supposed to have arisen via two independent interchromosomal duplications and several rounds of intrachromosomal duplications (9). The chromosome region 18q has often been reported as a region with high frequency of rearrangements (9, 13-16). The targets and functions of serpins are not fully understood. For most, the primary functions are regulation of proteolytic events associated with coagulation, fibrinolysis, apoptosis and inflammation, but alternative functions such as hormone transport and blood pressure regulation have been reported (17-24).

Although SCCA1 and SCCA2 are nearly identical they differ in their reactive site loops (FIGS. 2 and 3). SCCA1 inhibits the papain-like cystein proteinases cathepsin S, K, and L (25, 26) while SCCA2 inhibits the chymotrypsin-like serine proteinases cathepsin G and mast cell chymase (27). Studies of the reactive site loop (RSL) of SCCA1 show that the RSL is essential for cystein proteinase inhibition (28). The variable portion of the RSL dictates the specificity of the target proteinases shown by RSL swap mutants of SCCA1 and SCCA2 and single mutants (28, 29). It is likely that serpins utilize a common RSL-dependent mechanism to inhibit both serine and cystein proteinases.

The biological role of SCCA1 and SCCA2 are not fully understood. They are considered to be inhibitory serpins. Data suggest that SCCA are involved in apoptosis and expression makes cancer cells resistant to several killing mechanisms by inhibition of apoptosis (30).

SCCA1 and SCCA2 are detected in the cytoplasm of normal squamous epithelial cells (31, 33). The antigen, which appears in the serum of patients, may be a function of SCCA-overproduction by tumor cells and their normal turn over (34). It has been reported that the SCCA detected in serum by using antibody radioimmunology-assay or real-time-PCR, RT-PCR, is mainly SCCA2 (1, 35, 36) but other studies using PCR indicate that both antigens can be amplified and detected in patient samples (37).

Serum concentrations in patients with SCC are correlated to the clinical stage and to the degree of histological differentiation of the tumor (1). For cervical cancer several studies show a correlation between the pretreatment values and the clinical outcome (1, 38-43). Studies also show a correlation between high SCCA levels and tumor volume. Recurrence or progressive disease could be detected several months before clinical evidence (39). Similar results are seen for squamous cell carcinomas of the lung, vulva, head and neck and esophagus (1, 2, 44, 45). In all these studies, they have measured the total SCCA level.

SCCA's belong to the serpin family and it is likely that different forms of the serpins may be detected in tissue and in circulation. The general function of serpins is to regulate the activity of different proteolytic enzymes, and it may be speculated that also the SCCA1 and SCCA2 in tissues and serum may occur as the “free” serpin and as a complex with their target proteases. This would be similar to the serine protease PSA that in serum mainly is found as a complex with the serpin alfal-antichymotrypsin. The specific determination of SCCA1 and SCCA2 as well as the respective “free” and complex form of the respective serpin may also provide additional clinical information as compared to “total” SCCA.

### SUMMARY OF THE INVENTION

The present invention discloses the establishment of monoclonal antibodies capable of distinguishing between SCCA1 and SCCA2 as well as between the “free” and “total” amount of the respective serpin. In addition the invention describes the use of the established discriminatory antibodies for the design of immunoassays for determination of the total and “free” form of the SCCA1 and SCCA2 serpins, as well as the use of the immunoassays for diagnosis of cancer and detection of recurrent disease.

### BRIEF DESCRIPTION OF THE DRAWINGS

FIG. 1 In humans the serpins map to one of two chromosomal clusters. P16, P19 and ELNAH2 map to 6p25, whereas P18, Bomapin, PAI2, SCCA1, SCCA2, Headpin and Maspin map to 18q21.3;

FIG. 2-3 shows reactive site loops of SCCA1 and SCCA2. The nucleotide sequence for SCCA1 is shown in SEQ ID NO.: 1, and the amino acid sequence for SCCA1 is shown in SEG ID NO.: 3. The nucleotide sequence for SCCA2 is shown in SEQ ID NO.: 2, and the amino acid sequence is shown in SEQ ID NO.: 4;

FIG. 4 shows relative reactivity of SCC Mabs;

FIG. 5 shows relative reactivity of complex bound SCC Mabs;

FIG. 6 shows relative reactivity of “free” SCC Mabs;

FIG. 7 shows SCCA1 and SCCA2 in 20 samples of Squamous Cell Lung cancer, limited disease. The bars indicate the upper reference level of SCCA1 and SCCA2 respectively;

FIG. 8. SCCA1 and SCCA2 in Stage I cervical cancer. The bars indicate the upper reference level of SCCA1 and SCCA2 respectively;

FIG. 9. SCCA1 ad SCCA2 in Stage II cervical cancer. The bars indicate the upper reference level of SCCA1 and SCCA2 respectively.

FIG. 10. SCCA1 and SCCA2 in stage III-IV Cervical cancer. The bars indicate the upper normal levels;

FIG. 11 is a graph showing the amount of SCCA1 and SCCA2 in Patient No. 53;

FIG. 12 is a graph showing the amount of SCCA1 and SCCA2 in Patient No. 29;

FIG. 13 is a graph showing the amount of SCCA1 and SCCA2 in Patient No. 83;

FIG. 14 is a graph showing the amount of SCCA1 and SCCA2 in Patient No. 70;

FIG. 15 is a graph showing the amount of SCCA1 and SCCA2 in Patient No. 48; and

FIG. 16 is a graph showing the amount of SCCA1 and SCCA2 in Patient No. 45.

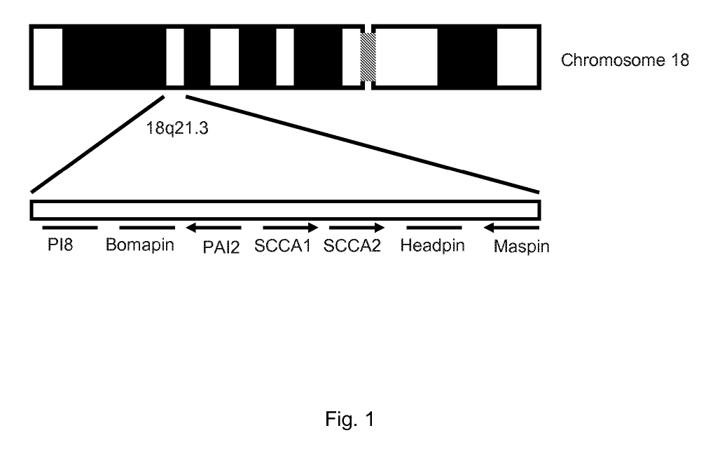

### DETAILED DESCRIPTION OF THE INVENTION

Establishment of monoclonal antibodies against epitopes of SCCA1 and SCCA2, as well as Pan SCCA exposed and hidden in the serine protease complex of the SCCA's, respectively, made it possible to design specific immunoassays for determination of the respective form of SCCA. Furthermore methods for diagnosis of cancer using the specific immunoassays are disclosed within the present invention.

### EXAMPLE 1

### Production of Recombinant SCCA

1.1 Cloning of SCCA

mRNAs from the cell-lines Caski (cervix), C-4I (cervix), A549 (lung), and RPMI2650 (pharynx) were prepared using QuickPrep Micro mRNA Purification kit (Pharmacia) and cDNA was prepared using First-Strand cDNA Synthesis kit (Pharmacia). A 1218 bp DNA fragment covering the coding sequence of SCCA was amplified by PCR in a 100 μl reaction containing 10 mM Tris-HCl pH 8.85, 25 mM KCl, 5 mM (NH₄)₂SO₄, 2 mM MgSO₄ (Boehringer), 0.2 mM dNTP (Pharmacia), 10 μM SCCA 1-7F (DNA sequences for all primers are shown in Table 1), 10 μM SCCA 391-397B, 2 μl cDNA and 2.5 U Pwo-polymerase (Boehringer). After denaturing samples for 5 min at 96° C., a total of 30 cycles were performed, each consisting of denaturation for 15 sec at 96° C., annealing for 15 sec at 60° C., and extension for 30 sec at 72° C. The PCR reaction was completed by a final extension for 10 min at 72° C.

Detection of SCCA1 and SCCA2

Presence of SCCA1 in PCR products were detected by cleavage with restriction enzyme SacII, resulting in two fragments, 245 and 973 bp, respectively, or by SCCA1-specific PCR using the primers SCCA1-7F and SCCA1 323-329B in a standard PCR reaction (75 mM Tris-HCl pH 8.8, 20 mM (NH₄)₂SO₄, 0.010% Tween 20, 2 mM MgCl₂, 0.2 mM dNTP, 10 μM of each primer, template, and 0.025 U/μl reaction Taq Polymerase; after denaturing samples for 5 min at 96° C. a total of 30 cycles were performed, each consisting of denaturation for 15 sec at 96° C., annealing for 15 sec at optimal annealing temperature, and extension for 30 sec at 72° C. The PCR reaction was completed by a final extension for 10 min at 72° C.), Ta=50° C., resulting in a 997 bp fragment. Presence of SCCA2 were detected by standard PCR using SCCA 1-7F and a SCCA2-specific primer, SCCA2 357-363B, Ta=60° C., giving a 1090 bp fragment.

Cloning

PCR-products were cloned using PCR-Script Amp cloning kit (Stratagene). Colony screenings were performed by PCR as described in 1. 2. Plasmid-DNAs were prepared from selected clones containing SCCA1 or SCCA2 using Wizard Plus Minipreps DNA Purification System (Promega).

DNA Sequencing

Clones were sequenced using ABI Prism BigDye Terminator Cycle Sequencing (PE Biosystems). Samples were run on an ABI Prism 310.

Recloning

Selected clones were recloned into the expression vector pGEX-6P-3 (Pharmacia). Fragments were excised from the PCR-Script Amp vector using BamHI and XhoI and ligated into the expression vector in a 10 μl reaction containing 1xOPA, 1 mM ATP, 50 ng cleaved vector, SCCA insert corresponding to a moles-of-ends vector:insert ratio of 1:5-1:8, and 7.5-10 U T4DNAligase (all from Pharmacia). Reaction tubes were incubated at 10° C. overnight and inactivated for 10 min at 65° C. 2-4 μl of the reaction was transformed into E. Coli JM109 (46). Plasmid-DNAs from selected clones were then transformed into E. Coli BL21 for protein expression.

Maintenance of Cloned Gene

Plasmid-DNA (pGEX-6P-3 containing the SCCA1/A2 fusion gene) in a 10 mM Tris-HCl (pH 8.0) buffer solution is stored in −80° C. For resuming protein expression, plasmid-DNA is transformed into competent E. coli BL21 according to Sambrook et al. (p 1.82-1.84 in ref. 46). For preparation of more plasmid-DNA, transformation into E. Coli JM109 is preferred.

1.1.2 Protein Expression and Purification

Protein Expression

Expression conditions were determined by small-scale preparations. For large scale expression 500 ml cultures of 2xYT and 100 μg of ampicillin/ml were inoculated with 5 ml over-night culture and grown at 37° C. Protein expression was induced at OD₆₀₀=0.5-1.3 by adding IPTG to a final concentration of 0.1 mM.

Protein Purification

Cells were harvested by centrifugation for 10 min at 2000 g, washed with 50 ml TE pH 8.0, and dissolved in 3 ml TE/g bacterial pellet. Lysozyme was added to a final concentration of 800 μg/g pellet and the mixtures were incubated on ice for 30-60 min and then frozen over night at −70° C. Magnesium chloride and DNase were added to a final concentration of 12 mM and 20 μg/g pellet, respectively. After incubation on ice for 30 min, samples were centrifuged for 30 min at 40000 g. To each supernatant 0.5 ml of 50% Glutathione Sepharose (Pharmacia) was added and incubated for 30 min-2 h at room temperature with gentle agitation. The slurry was washed 5-7 times using 1×PBS. GST-SCCA fusion protein was eluated using 0.5-1 ml Reduced Glutathione (Pharmacia) and incubated for 30-60 min at room temperature or over-night at 4° C., all with gentle agitation. SCCA protein was eluated by cleavage in between GST and SCCA. 0.48 ml cleavage buffer (50 mM Tris-HCl pH 7.0, 150 mM NaCl, 1 mM EDTA, 1 mM DTT) and 20 μl PreScission protease were added and samples were incubated at 4° C. with gentle agitation for 4 h or over-night. Proteins were analyzed on SDS-PAGE by Phast-system (Pharmacia).

### EXAMPLE 2

### Establishment of Hybridomas and Monoclonal Antibodies

2. 1 Immunization and Primary Selection of Anti SCCA Hybridomas

Polyclonal antisera reactive with SCC antigen were obtained by subcutaneous immunization of rabbits with recombinant SCC antigen and collection of immune sera according to standard procedures. The titer of the polyclonal antisera was tested by determination of the reactivity of the antisera with biotinylated SCCA2 and SCCA1 immobilized in streptavidin plates (Labsystems Oy, Helsinki, Finland). The recombinant SCCA2 and SCCA1 were biotinylated with Biotin-N-succinimide caproate ester according to standard procedures.

Monoclonal antibodies reactive with SCCA1 and SCCA2 were obtained by immunization of Balb/c mice intraperitoneally with 10-50 μg of recombinant SCCA in Ribi adjuvant. After the immunization and 2-4 booster doses during 60-90 days spleen cells from the immunized mice were fused with P3×63Ag 8 myeloma cells as described.

Hybridomas producing antibodies reacting with SCCA1 and/or SCCA2 were selected by ELISA screening of hybridoma supernatants in microtitre wells coated with affinity purified polyclonal antiserum against mouse IgG+M, (Jackson Immuno Res Lab, US). The wells were then incubated with SCCA antigen, and after washing, the bound antigen was detected by incubation with polyclonal Rabbit Anti SCC and HRP labeled Swine Anti Rabbit Ig (Dako AS, Copenhagen, Denmark).

2. 2. Reactivity of Selected Hybridomas with SCC Antigens

The reactivity of the established hybridomas was tested in an ELISA similar to the screening procedure. Briefly the monoclonal antibodies produced by the hybridomas were immobilized in microtitre plates coated with polyclonal antiserum against mouse IgG+M (Jackson Immuno Res Lab, US). The wells were then incubated with 50 μL of the different recombinant SCC antigens (SCCA1, SCCA2, SCCA1/A2 and SCCA2/A1 fusion protein) in PBS 1% BSA for 1 h, after washing the plates were incubated with 100 μL rabbit anti-SCC diluted 1/5000 in PBS-1% BSA and incubated for additional 1 h. The bound rabbit Anti-SCC was then detected by incubation with HRP—Swine anti Rabbit Ig and visualized with OPD substrate and determination of OD at 450 nm.

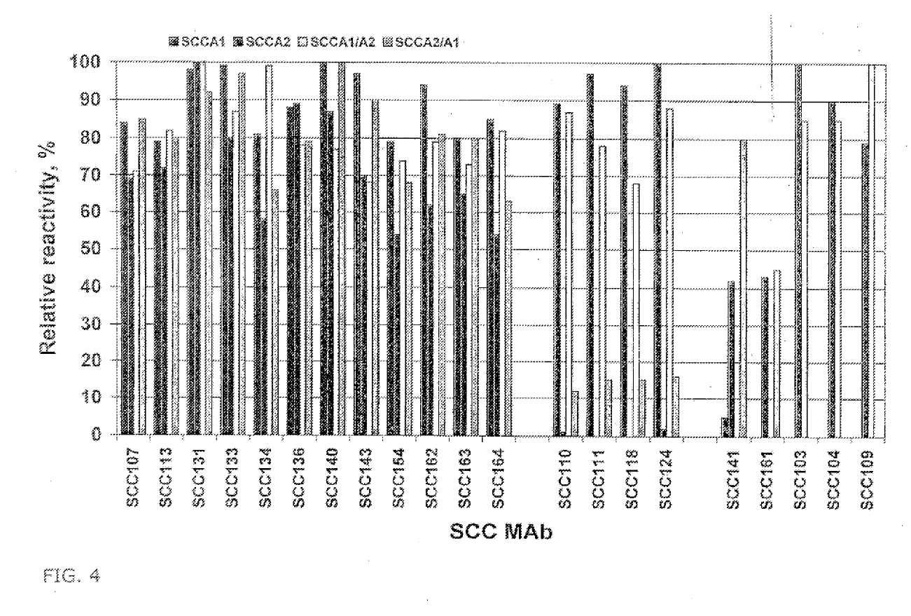

In FIG. 4 the reactivity of selected hybridomas are shown. They are also evident from the Table 1 below

TABLE 1

| SCC Mab | SCCA1 | SCCA2 | SCCA1/A2 | SCCA2/A1 |
|---|---|---|---|---|
| SCC107 | 84 | 69 | 71 | 100 |
| SCC113 | 79 | 72 | 82 | 100 |
| SCC131 | 98 | 100 | 100 | 92 |
| SCC133 | 99 | 80 | 87 | 97 |
| SCC134 | 81 | 58 | 99 | 66 |
| SCC136 | 88 | 89 | 78 | 79 |
| SCC140 | 100 | 57 | 77 | 100 |
| SCC143 | 97 | 70 | 68 | 90 |
| SCC154 | 79 | 54 | 74 | 68 |
| SCC162 | 94 | 62 | 79 | 81 |
| SCC163 | 80 | 65 | 73 | 80 |
| SCC164 | 85 | 54 | 82 | 63 |
| SCC110 | 89 | 1 | 87 | 12 |
| SCC111 | 97 | 0 | 78 | 15 |
| SCC118 | 94 | 0 | 68 | 15 |
| SCC124 | 100 | 2 | 88 | 16 |
| SCC141 | 5 | 42 | 0 | 80 |
| SCC161 | 0 | 43 | 0 | 45 |
| SCC103 | 0 | 100 | 85 | 0 |
| SCC104 | 0 | 90 | 85 | 0 |
| SCC109 | 0 | 79 | 100 | 0 |

2.3 Selection of Monoclonal Antibodies Discriminating Between Free and Complex-Bound SCCA

MAb reacting with epitopes exposed in SCCA-protease complexes as well as Mab reacting with epitopes “hidden” in the serpin-protease complex were selected by determination of binding to SCCA-protease complex and to “free” SCCA.

2.3.1 Establishment of SCCA-Protease Complexes

Complex binding of SCCA to target proteases was performed by mixing 2 μg of SCCA-protein with 0.5 μg of Cathepsin G (Biodesign Int.) or 0.5 μg of 0.9 μg Cathepsin L (Calbiochem) in 1×PBS buffer in a total volume of 4.5 μl. Samples were incubated at 37° C. for 30 minutes. To each sample, 0.5 μl of 10xComplex-buffer (20% SDS, 140 mM Mercaptoethanol, bromophenolblue) was added. Samples were incubated for 3 minutes at 95° C. and analyzed on a 12.5% SDS-PAGE-gel.

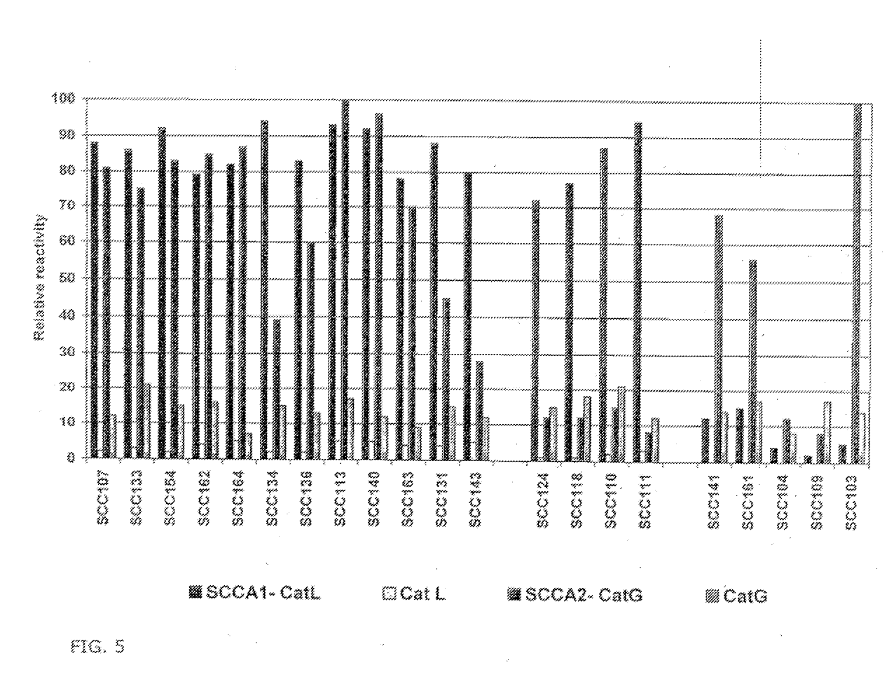

The reactivity of complex binding is evident from the Table 2 below and FIG. 5.

2.3.2 Reactivity with SCCA-Protease Complexes

MAb that recognized epitopes that did not interfere with complex formation between SCCA1 and Cathepsin L and SCCA2 and Cathepsin G, respectively, was detected by preincubation of antibodies recognizing epitopes located within Exon 2-7 of SCCA1 and SCCA2 respectively, and then determination of complex formation in ELISA assays as described.

Based on the capability to inhibit the complex formation between SCCA1 and Cathepsin L and SCCA2 and Cathepsin G, respectively it was deduced that a number of antibodies recognized epitopes that were not influenced by the complex formation between the serpins and the target proteases. In FIG. 5, as well as Table 2 below the reactivity of antibodies with serpin-proteases are shown.

TABLE 2

| SCC Mab | SCCA1-CatL | Cat L | SCCA2-CatG | CatG |
|---|---|---|---|---|
| SCC107 | 88 | 2 | 81 | 12 |
| SCC133 | 86 | 3 | 75 | 21 |
| SCC154 | 92 | 2 | 83 | 15 |
| SCC162 | 79 | 4 | 85 | 16 |
| SCC164 | 82 | 5 | 87 | 7 |
| SCC134 | 94 | 2 | 39 | 15 |
| SCC136 | 83 | 2 | 60 | 13 |
| SCC113 | 93 | 5 | 100 | 17 |
| SCC140 | 92 | 5 | 96 | 12 |
| SCC163 | 78 | 4 | 70 | 9 |
| SCC131 | 88 | 4 | 45 | 15 |
| SCC143 | 80 | 5 | 28 | 12 |
| SCC124 | 72 | 1 | 12 | 15 |
| SCC118 | 77 | 1 | 12 | 18 |
| SCC110 | 87 | 2 | 15 | 21 |
| SCC111 | 94 | 3 | 8 | 12 |
| SCC141 | 12 | 0 | 68 | 14 |
| SCC161 | 15 | 0 | 56 | 17 |
| SCC104 | 4 | 0 | 12 | 8 |
| SCC109 | 2 | 0 | 8 | 17 |
| SCC103 | 5 | 0 | 100 | 14 |

The antibodies described in 2.3.1., which reacted with epitopes located in Exon 8 inhibited complex formation between the respective serpin and its protease. It may be deduced that these antibodies recognized “hidden” epitopes.

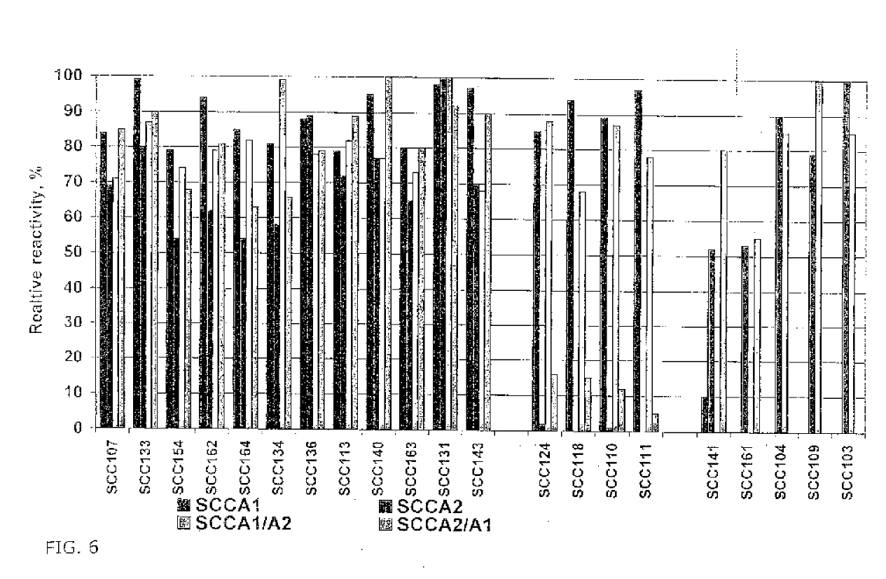

Complexes to “free” SCCA is shown in Table 3 below, as well as inj FIG. 6.

TABLE 3

| SCC Mab | SCCA1 | SCCA2 | SCCA1/A2 | SCCA2/A1 |
|---|---|---|---|---|
| SCC107 | 84 | 69 | 71 | 85 |
| SCC133 | 99 | 80 | 87 | 90 |
| SCC154 | 79 | 54 | 74 | 68 |
| SCC162 | 94 | 62 | 79 | 81 |
| SCC164 | 85 | 54 | 82 | 63 |
| SCC134 | 81 | 58 | 99 | 66 |
| SCC136 | 88 | 89 | 78 | 79 |
| SCC113 | 79 | 72 | 82 | 89 |
| SCC140 | 95 | 77 | 77 | 100 |
| SCC163 | 80 | 65 | 73 | 80 |
| SCC131 | 98 | 100 | 100 | 92 |
| SCC143 | 97 | 70 | 68 | 90 |
| SCC124 | 85 | 2 | 88 | 16 |
| SCC118 | 94 | 0 | 68 | 15 |
| SCC110 | 89 | 1 | 87 | 12 |
| SCC111 | 97 | 0 | 78 | 5 |
| SCC141 | 10 | 52 | 0 | 80 |
| SCC161 | 0 | 53 | 0 | 55 |
| SCC104 | 0 | 90 | 85 | 0 |
| SCC109 | 0 | 79 | 100 | 0 |
| SCC103 | 0 | 100 | 85 | 0 |

2.3.3 Summary of Reactivity of Established MAb

The reactivity of the established monoclonal antibodies against different forms of SCCA are summarized in Table 4.

TABLE 4

| Group A | | | | | Group B | | Group C | | | |
|---|---|---|---|---|---|---|---|---|---|---|
| PAN SCC MAb | | | | | SCCA1 MAb | | SCCA MAb | | | |
| A1a | A1b | A2 | A3a | A3b | B1 | B2 | C1a | C1b | C2a | C2b |
| SCC | SCC | SCC | SCC | SCC | SCC | K134 | SCC | SCC | SCC | SCC |
| 107 | 134 | 113 | 140 | 131 | 124 | | 141 | 161 | 103 | 104 |
| SCC | SCC | | SCC | SCC | SCC | K135 | | | | SCC |
| 119 | 136 | | 163 | 143 | 118 | | | | | 109 |
| SCC | | | | | SCC | | | | | K122 |
| 123 | | | | | 110 | | | | | |
| SCC | | | | | SCC | | | | | |
| 128 | | | | | 111 | | | | | |
| SCC | | | | | | | | | | |
| 133 | | | | | | | | | | |
| SCC | | | | | | | | | | |
| 154 | | | | | | | | | | |
| SCC | | | | | | | | | | |
| 162 | | | | | | | | | | |
| SCC | | | | | | | | | | |
| 164 | | | | | | | | | | |

Groups A1b and A3b react preferentially with “Free” SCC; Groups C1a and C2a recognize “Total SCCA2”, while Group C1b and C2b recognize only “Free SCCA2”

2.4 Production of Discriminatory Monoclonal Antibodies

Monoclonal antibodies were produced by in vitro cultivation of the hybridoma clones by inoculation of 10⁴ cells/mL in DMEM, 5% Fetal Calf Serum in roller bottles and allowed to grow for 10-14 days. The monoclonal antibodies were then purified from the culture medium by Protein A (Bioprocessing Ltd, Durham, UK) affinity chromatography according to the manufacturers recommendation.

### EXAMPLE 3

### Establishment of Immunoassays

Using the established monoclonal antibodies and recombinant proteins it was possible to develop immunoassays for specific determination total SCCA and total “free” SCCA, and assays specific for total SCCA1 and “free” SCCA1 as well as assays for specific determination of total SCCA2 and “free” SCCA2, respectively.

3. 1. Immunoassays for Determination of Total SCCA

3.1.1 Immunoassays for Determination of “Total SCCA”

Assays specific for SCCA, i.e the total of “free” SCCA1, “free” SCCA2, complexed SCCA1 and complexed SCCA2 were designed by using antibodies among Ala (Table 1) in combination with antibodies from Groups A2 or A3a.

In the preferred configuration antibody SCC113 was used as catching antibody and SCC107 as detecting antibody.

SCC113 MAb was biotinylated with BiotinNHRS caproate ester, Sigma Chemical Co, US, using standard procedures, and used as catching antibody. SCC107 MAb were conjugated with HRP according to a modification of the Nakone procedure.

The biotinylated SCC113 MAb and HRP conjugated SCC107 MAb were used in one-step EIA according to the following protocol.

Assay Procedure:

1. Add 25 μL of SCCA recombinant antigen (0-50 μg/L in PBS, 60 g/L BSA, pH 7.2)+100 μL of Biotin SCC113 MAb, 1 μg/mL and HRPSCC107, 1 μg/mL in Assay Buffer in Streptavidin coated microtiter plates, Labsystems Oy, Helsinki, Finland.
2. Incubate for 1 h±10 min with shaking
3. Wash 6 times with 5 mM Tris buffer, 0.05% Tween 40, pH 7.75.
4. Add 100 μL TMB, ELISA Technology, US.
5. Incubate 30 min±5 min
6. Determine OD 620 nm in ELISA reader.

Dose-response curves for free and complex SCCA1 and SCCA2 antigens revealed that the assay recognized all forms of SCCA.

3. 2. Assays for Specific Determination of SCCA1

3. 2. 1 Assays for Total SCCA1

Assays specific for total SCCA1, i.e. Free and Complex SCCA1, without significant reactivity with SCCA2 were designed by using antibodies of Group B1 in combination with antibodies from Group A1a, A2 or A3a. In the preferred configuration SCC110 MAb was used as catching antibody and the SCC107 was used as detecting antibody.

SCC111 MAb was biotinylated with BiotinNHRS caproate ester (Sigma Chemical Co, US) using standard procedures, and used as catching antibody. SCC107 MAb was conjugated with HRP, Type V (Sigma Chemical Co, US), according to a modification of the Nakone procedure.

The biotinylated SCC111 MAb and HRP conjugated SCC107MAb were used in two-site EIA according to the following protocol.

Assay Procedure:

1. Add 50 μL of SCC recombinant antigen (0-100 μg/L in PBS, 60 g/L BSA, pH 7.2)+100 μL of Biotin SCC111 MAb, 2 μg/mL, in Assay Buffer in Streptavidin coated microtiter plates (Labsystems Oy, Helsinki, Finland).
2. Incubate for 1 h±10 min with shaking
3. Wash 3 times with 5 mM Tris buffer, 0.05% Tween 40, pH 7.75.
4. Add 100 μL HRP SCC107 MAb 2 μg/mL, in Assay Buffer.
5. Incubate for 1 h±10 min with shaking.
6. Wash 6 times with 5 mM Tris buffer, 0.05% Tween 40, pH 7.75.
7. Add 100 μL TMB, ELISA Technology, US
8. Incubate 30 min±5 min
9. Determine OD 620 nm in ELISA reader.

Based on the dose-response curves for SCCA1 and SCCA2 it was concluded that the assay according to example 3.2.1 recognized all forms of SCCA1 with a cross-reactivity of <5% for SCCA2.

3. 2. 2 Assays for “Free” SCCA1

Assays specific for “free” SCCA1, i.e. specific for uncomplexed SCCA1 without significant reactivity with complex SCCA1 or SCCA2 were designed by using antibodies of Group B2 in combination with antibodies of Group Ala. In the preferred configuration SCCK134 MAb was used as catching antibody and the SCC107 was used as detecting antibody.

SCCK134 MAb was biotinylated with BiotinNHRS caproate ester (Sigma Chemical Co, US) using standard procedures, and used as catching antibody. SCC107 MAb was conjugated with HRP, Type V (Sigma Chemical Co, US), according to a modification of the Nakone procedure.

The biotinylated SCCK134 MAb and HRP conjugated SCC107 MAb were used in two-site EIA according to the following protocol.

Assay Procedure:

1. Add 50 μL of SCC recombinant antigen (0-100 μg/L in PBS, 60 g/L BSA, pH 7.2)+100 μL of Biotin SCCK134MAb, 2 μg/mL, in Assay Buffer in Streptavidin coated microtitre plates (Labsystems Oy, Helsinki, Finland).
2. Incubate for 1 h±10 min with shaking
3. Wash 3 times with 5 mM Tris buffer, 0.05% Tween 40, pH 7.75.
4. Add 100 μL HRP SCC107MAb 2 μg/mL, in Assay Buffer.
5. Incubate for 1 h±10 min with shaking.
6. Wash 6 times with 5 mM Tris buffer, 0.05% Tween 40, pH 7.75.
7. Add 100 μL TMB, ELISA Technology, US
8. Incubate 30 min±5 min
9. Determine OD 620 nm in ELISA reader.

Based on the dose-response curves for SCCA1 and SCCA2 it was concluded that the assay according to example 3.2.2 recognized only “FREE” SCCA1 with a cross-reactivity of <5% for complex SCCA1 or SCCA2.

3. 3. Assays for Specific Determination of SCCA2

3. 3. 1 Assays for Determination of Total SCCA2

Assays specific for total SCCA2, i.e. free and complex SCCA2, without significant reactivity with SCCA1 were designed by using antibodies of Groups C1a or C2a in combination with antibodies of Group Ala. In the preferred configuration SCC103 MAb was used as catching antibody and the SCC107 was used as detecting antibody.

SCC103 MAb was biotinylated with BiotinNHRS caproate ester (Sigma Chemical Co, US) using standard procedures, and used as catching antibody. SCC107 MAb was conjugated with HRP, Type V (Sigma Chemical Co, US), according to a modification of the Nakone procedure.

The biotinylated SCC103 MAb and HRP conjugated SCC107 MAb were used in two-site EIA according to the following protocol.

Assay Procedure:

1. Add 50 μL of SCC recombinant antigen (0-100 μg/L in PBS, 60 g/L BSA, pH 7.2)+100 μL of Biotin SCC103 MAb, 2 μg/mL, in Assay Buffer in Streptavidin coated microtiter plates (Labsystems Oy, Helsinki, Finland).
2. Incubate for 1 h±10 min with shaking
3. Wash 3 times with 5 mM Tris buffer, 0.05% Tween 40, pH 7.75.
4. Add 100 μL HRP SCC107 MAb 2 μg/mL, in Assay Buffer.
5. Incubate for 1 h±10 min with shaking.
6. Wash 6 times with 5 mM Tris buffer, 0.05% Tween 40, pH 7.75.
7. Add 100 μL TMB, ELISA Technology, US
8. Incubate 30 min±5 min
9. Determine OD 620 nm in ELISA reader.

Based on the dose-response curves for SCCA1 and SCCA2 it was concluded that the assay according to example 3.3.1 recognized all forms of SCCA2 with a cross-reactivity of <5% for SCCA2.

3.3.2 Assays for “Free” SCCA2

Assays specific for “free” SCCA2, i.e. non-complexed SCCA2, without significant reactivity with SCCA2-protease complex or SCCA1 were designed by using antibodies from Group C2b in combination with antibodies of Group A1a In the preferred configuration SCC104 MAb was used as catching antibody and the SCC107 was used as detecting antibody.

SCC104 MAb was biotinylated with BiotinNHRS caproate ester (Sigma Chemical Co, US) using standard procedures, and used as catching antibody. SCC107 MAb was conjugated with HRP, Type V (Sigma Chemical Co, US), according to a modification of the Nakone procedure.

The biotinylated SCC104 MAb and HRP conjugated SCC107 MAb were used in two-site EIA according to the following protocol.

Assay Procedure:

1. Add 50 μL of SCC recombinant antigen (0-100 μg/L in PBS, 60 g/L BSA, pH 7.2)+100 μL of Biotin SCC104MAb, 2 μg/mL, in Assay Buffer in Streptavidin coated microtiter plates (Labsystems Oy, Helsinki, Finland).
2. Incubate for 1 h±10 min with shaking
3. Wash 3 times with 5 mM Tris buffer, 0.05% Tween 40, pH 7.75.
4. Add 100 μL HRP SCC107 MAb 2 μg/mL, in Assay Buffer.
5. Incubate for 1 h±10 min with shaking.
6. Wash 6 times with 5 mM Tris buffer, 0.05% Tween 40, pH 7.75.
7. Add 100 μL TMB, ELISA Technology, US
8. Incubate 30 min±5 min
9. OD 620 nm in ELISA reader.

Based on the dose-response curves for SCCA1 and SCCA2 it may be concluded that the immunoassay according to 3.3.2 recognized only “free” SCCA2 with a cross-reactivity of <5% for complex SCCA2 or SCCA1

### EXAMPLE 4

Diagnosis of cancer using immunoassays discriminatory for “free” SCCA.

The immunoassays according to Example 3 were used to determine different forms of SCCA in healthy individuals and in patients with squamous cell carcinoma.

All assays showed discrimination between healthy individuals and cancer patients as expected. However, the discriminatory ratio between healthy and cancer subjects were higher for assays determining SCCA2, which was further improved by determination of the ratio between free and complex SCCA2 and between SCCA2 and SCCA1.

SCCA isoforms were determined in 50 blood donors and in 50 healthy subjects aged 50-65 Years in order to determined upper normal level. SCCA isoforms were also determined in the assays according to Example 3 in 94 samples for females diagnosed with cervical cancer and in 20 individuals with squamous cell lung cancer.

### EXAMPLE 4.1

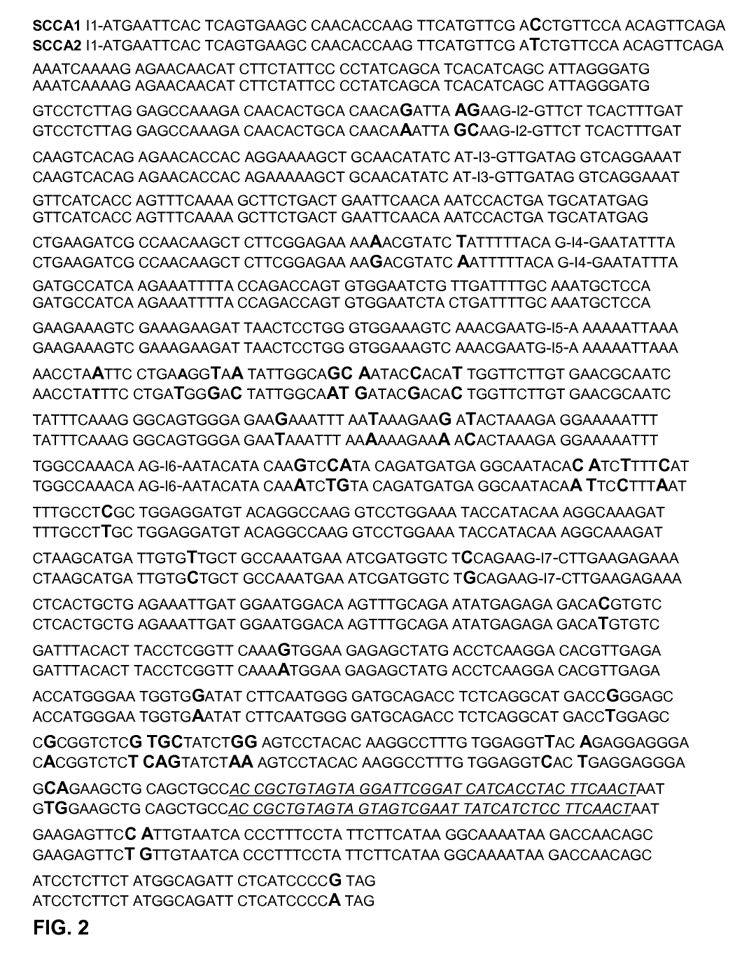

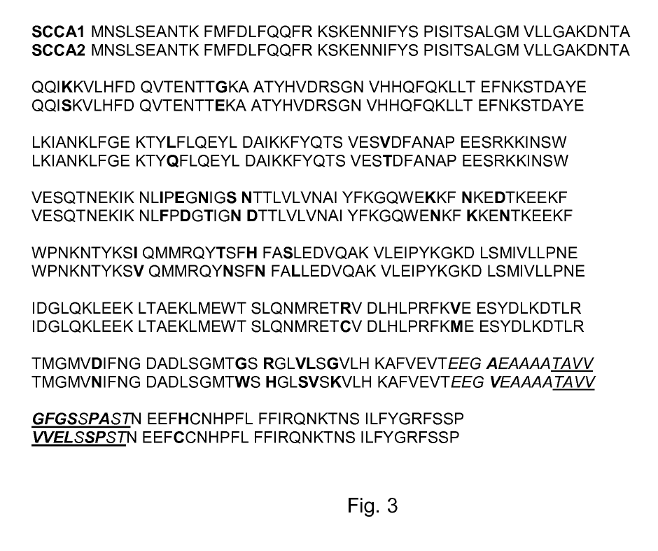

The results for Squamous cell lung cancer are shown in FIG. 2. SCCA1 was above upper normal level in 14 patients while SCCA2 was elevated in 18 patients. The level of SCCA2 was also relatively higher as compared to SCCA1 and thus improving the discrimination between healthy subjects and individuals with malignant disease

### EXAMPLE 4.2 SCCA in Cervical Cancer

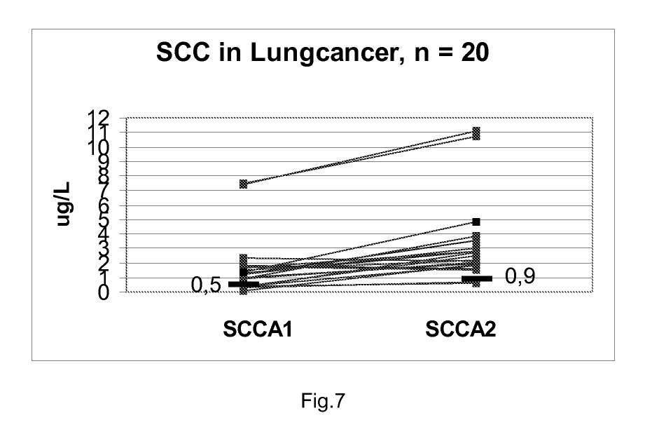

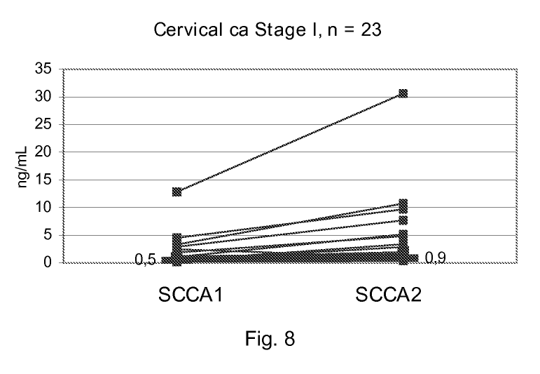

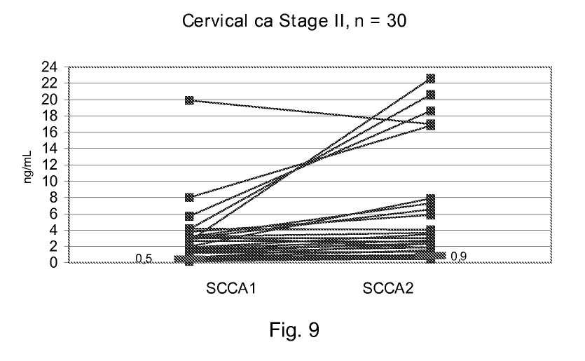

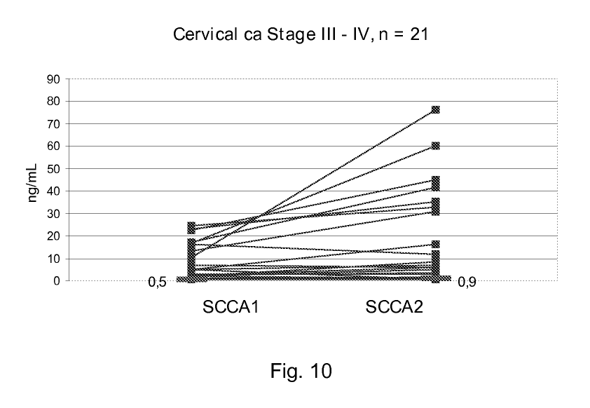

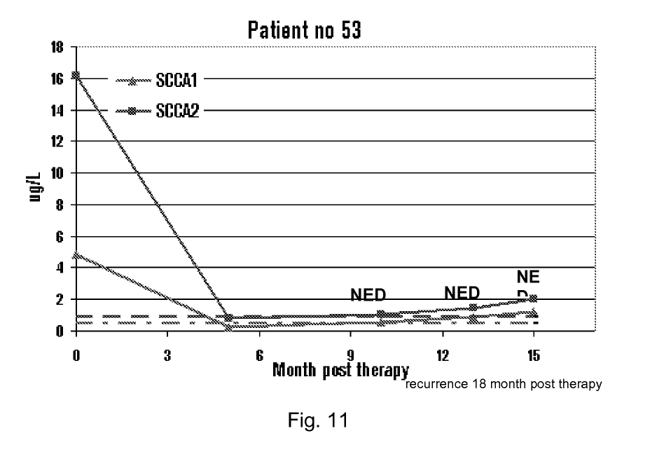

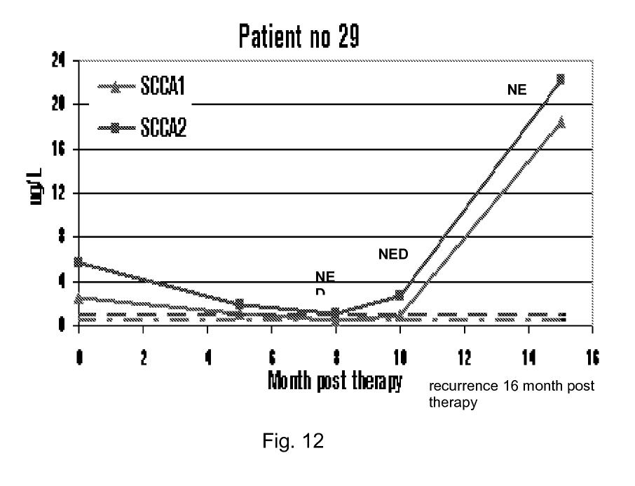

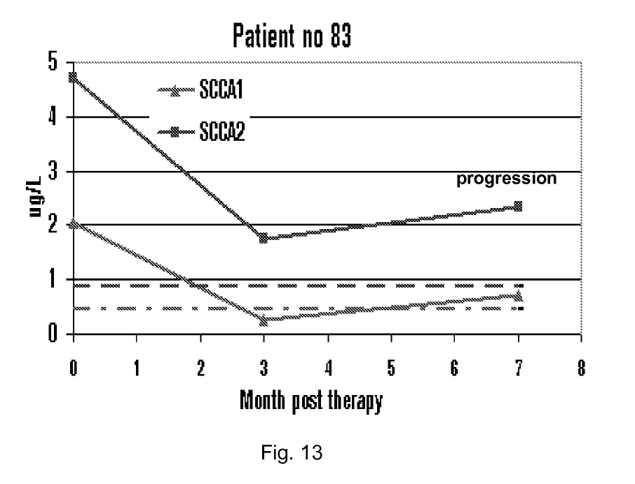

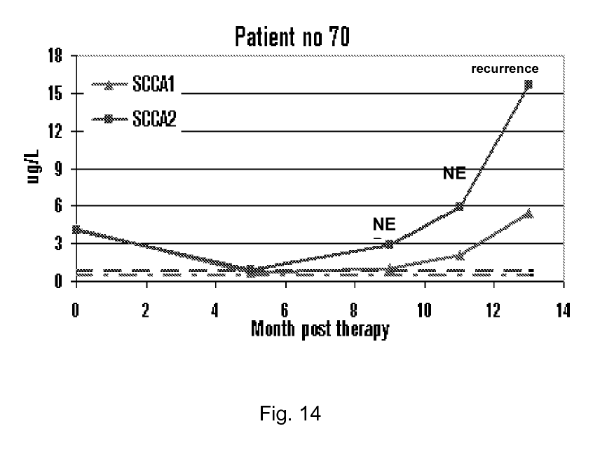

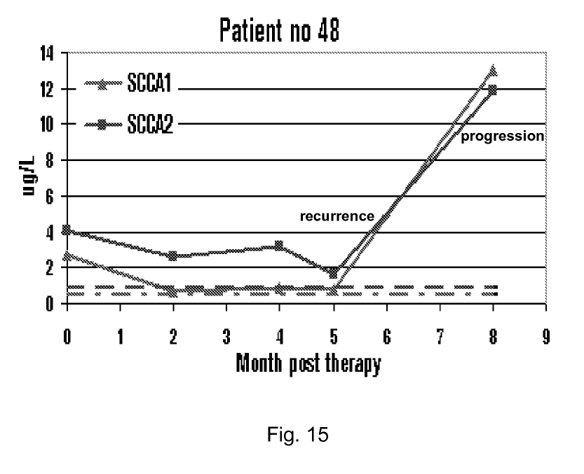

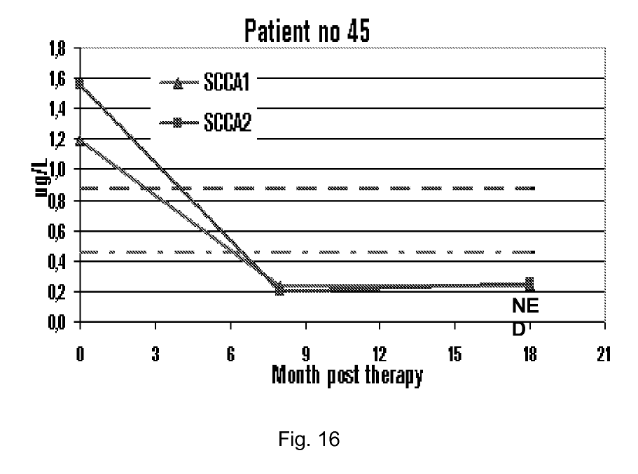

The levels of SCCA1 and SCCA2 in pretherapy samples from females with cervical cancer are shown in FIGS. 7-10. SCCA2 was in most cases relatively higher elevated as compared to SCCA1. Thus increasing the discrimination between healthy subjects and individuals with cervical cancer.

### EXAMPLE 4.3 SCCA1 and SCCA2 in Therapy Monitoring of Cervical Cancer

SCCA1 and SCCA2 were determined using assays according to Example 3 in 6 patients during therapy monitoring. Both SCCA1 and SCCA2 followed the clinical course of the disease, and detected recurrent disease prior to clinical manifestation of disease in 4/4 patient. However in the patients the relative increases of SCCA2 was higher compared to SCCA1 thus providing an early signal of recurrent disease. In the patient with NED both SCCA1 and SCCA2 were normalized after the therapy.

Recurrent disease was detected in patient 53 18 months post therapy. The recurrence was indicated by elevated SCCA1 and SCCA2, but SCCA2 responded earlier and showed a higher level as indication of the recurrence as compared to SCCA1.

In patient 29 recurrence was clinically detected 16 months post therapy, which was indicated by elevated SCCA2 from 8 months post therapy, which was 2-3 months earlier than SCCA1.

Patient 83 showed progressive disease 7 months post initial therapy. SCCA2 was never normalized, while SCCA1 normalized 3 months after initial therapy and then maws marginally elevated at the time of clinical diagnosis of progressive disease.

Recurrent disease was clinically diagnosed in patient 70 after 13 months. SCCA2 stated to increase between 5-6 months post therapy. SCCA1 also was slightly elevated 9 months post therapy and afterwards followed the clinical course. However the SCCA2 more clearly indicated the recurrent disease 5-7 months before clinical diagnosis.

SCCA2 levels never normalized in patient 48 suggesting recurrence and progressive disease already 2 months post therapy. SCCA1 was on the upper normal level until 5 months post therapy before increasing.

Patient 45 responded to the treatment and no evidence of disease was noticed after the therapy. This was indicated by both SCCA1 and SCCA2 as the levels were normalized and stayed in the normal range.

Both SCCA1 and SCCA2 followed the clinical course of the disease. However SCCA2 provided earlier and more distinct response of recurrent disease as compared to SCCA1.

We claim:

1-27. (canceled)

28. A monoclonal antibody which selectively binds only free SCCA2 molecules, excluding complexed SCCA2 molecules, for diagnosing cancer or the detection of recurrent cancer disease.

29. A monoclonal antibody according to claim 28, wherein the antibody has been produced by hybridoma, said hybridoma cells deriving from spleen cells from a mammal which have been spliced with myeloma cells.

30. Immunoassays based on antibodies according to claim 28 for specific determination of free SCCA2 molecules, excluding complexed SCCA2 molecules.

31. A kit for diagnosing squamous cell carcinomas or detecting recurrent squamous cell carcinoma disease, whereby the kit comprises monoclonal antibodies capable of distinguishing between free and total SCCA2 and providing a ratio between said free and total SCCA2, which ratio can be compared with a ratio obtained in healthy subjects, a differing ratio indicating presence or recurrence of squamous cell carcinomas.

32. A kit for diagnosing squamous cell carcinomas or detecting recurrent squamous cell carcinoma disease, whereby the kit comprises hybridomas which produce monoclonal antibodies capable of distinguishing between free and total SCCA2 and providing a ratio between said free and total SCCA2, which ratio can be compared with a ratio obtained in healthy subjects, a differing ratio indicating presence or recurrence of squamous cell carcinomas.
